(19) United States
(12) Patent Application Publication
(10) Pub. No.: US 2008/0052791 A1
(43) Pub. Date: Feb. 28, 2008

(54) DROUGHT TOLERANCE IN PLANTS

(75) Inventors: Ping Xu, Ardmore, OK (US); Jonathan Mannas, Ardmore, OK (US); Marilyn J. Roossinck, Springer, OK (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Samuel Roberts Noble Foundation

(21) Appl. No.: 11/668,356

(22) Filed: Jan. 29, 2007

Related U.S. Application Data
(60) Provisional application No. 60/763,668, filed on Jan. 30, 2006.

(51) Int. Cl. C12N 15/00

(52) U.S. Cl. 800/278

(57) ABSTRACT

The invention provides methods for enhancing drought tolerance in a plant, by infecting the plant with a plant virus, or infectious material derived from a plant virus. The plant thereby displays fewer, less severe, and/or delayed symptoms of dehydration. The reduction in symptoms allows for improved plant growth.

This application claims the priority of U.S. Provisional Patent Application 60/763,668, filed Jan. 30, 2006, which is herein incorporated by reference in its entirety.

### BACKGROUND OF THE INVENTION

1. Field of the Invention

The present invention relates generally to the field of agriculture. More particularly, the invention relates to the use of plant viruses for improving drought tolerance in plants.

2. Description of Related Art

Plant viruses are often discovered and studied as pathogenic parasites that cause disease in plants, including agricultural crops. Numerous plant viruses and virus strains are known, and may be classified based on the structure of their encoding nucleic acids (e.g. DNA or RNA), its polarity (positive or negative sense), genome strandedness, genome structure (e.g. monopartite, bipartite), and on capsid structure (e.g. icosahedral, rigid rod, flexuous rod, etc.). For a positive-sense virus, the genome and the mRNA correspond to the same nucleic acid strand, while for a negative-stranded virus, the genome corresponds to the complement of the viral mRNA. The majority of plant viruses are positive-sense single-stranded RNA viruses. Important groups of plant viruses include the Bromoviruses, Potyviruses, Tobamoviruses, and Caulimoviruses, among others. Virus satellites and viroids are also known, which are infectious nucleic acids that depend on the function of a structurally unrelated helper virus for their replication.

Infection of a plant host by a compatible virus, and subsequent replication and spread in the plant often results in symptoms including altered leaf color (e.g. chlorosis, mosaic, or vein clearing), stunting, reduced yield, and altered morphology of plant structures, among others. Such viruses may spread systemically throughout a plant from the initial infection site. Incompatible viruses, in contrast, replicate and/or spread poorly, if at all, in a plant. Some plant hosts are asymptomatic, and may serve as reservoirs leading to infections that are only recognized on symptomatic hosts. It is widely believed that viruses are harmful to their hosts because they use host resources to support their own reproduction, and may interfere with plant metabolism such as chloroplast development or function.

Plant responses to biotic and abiotic stress, including abiotic stress such as drought (e.g. Bohnert et al., 1995; Bray, 1997), are complex, and are thought to include multiple signaling pathways including changes in levels of protective osmolytes and antioxidants, stress proteins, and changes in levels of plant hormones, among other responses. TMV infection of host plants has been reported to result in increased abscisic acid (ABA) levels in infected tobacco plants (Whenham et al., 1986).

For some bacterial, fungal, and animal viruses mutualistic relationships have been described. For instance, some ascoviruses of wasps can be mutualistic depending on the specific virus and wasp strains (Stasiak et al., 2005). In addition, the polydnaviruses of braconid wasps are required for survival of the parasitoid wasps in their caterpillar hosts (Webb, 1998). Several dsRNA mycoviruses are known in the fungus Ustilago maydis. For instance, the virus UMV4 encodes KP4 killer toxin, and enables its host, U. maydis strain P4, to outcompete other uninfected U. maydis strains and related fungi (Gage et al., 2001). Human endogenous retroviruses may protect human tissue from infection with the exogenous retrovirus Spleen necrosis virus and may protect a developing fetus (Ryan, 2004). Certain mutualistic symbioses have also been reported in plants (e.g. Schardl et al., 2004). However, mutualistic interactions between plant viruses and their hosts have not been previously described, nor has the use of virus infection for improving drought tolerance in plants. Such interactions may have important agricultural applications, especially as drought is one of the most limiting factors in crop production worldwide (Wollenweber et al., 2005).

### SUMMARY OF THE INVENTION

In one aspect, the invention provides a method for enhancing drought tolerance in a plant comprising: (a) identifying a plant in need of increased drought tolerance; and (b) infecting the plant with a plant virus or infectious plant viral material that increases the drought tolerance in the plant as compared to an uninfected plant of the same genotype when grown under the same conditions. In the method the virus may or may not produce a disease symptom in the plant. In one embodiment, step (a) comprises identifying a plant exhibiting drought stress. In another embodiment, step (a) comprises identifying a plant in a growing environment under drought conditions.

A plant used with the invention may be any plant and in certain embodiments may be defined as a rice, beet, cucumber, zucchini, watermelon, tomato or Nicotiana sp. In particular embodiments, a virus used with the invention may be Cucumber Mosaic Virus (CMV), and may be selected from the group consisting of: Cucumoviruses, Tobamoviruses, Tobraviruses, and Bromoviruses, including a virus is selected from the group consisting of Cucumber Mosaic Virus, Tobacco Mosaic Virus, Tobacco Rattle Virus, and Brome Mosaic Virus. An infectious material may be used in accordance with the invention, such as a plant virus virion, an RNA transcript, or a plant virus cDNA clone. In certain embodiments, a plant may be infected with a virus by a method selected from the group consisting of: mechanical inoculation, spraying, injection, infiltration, grafting, seed or pollen transmission, and vector transmission. Virus infection may comprise mechanical inoculation.

In another aspect, the invention provides a method of reducing osmotic stress damage in a plant, comprising the steps of: (a) identifying a plant under osmotic stress; and (b) infecting the plant with a plant virus.

The foregoing has outlined certain features and technical aspects of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1: Comparison of drought symptoms in mock- and CMV-infected rice plants at 7 days after watering.

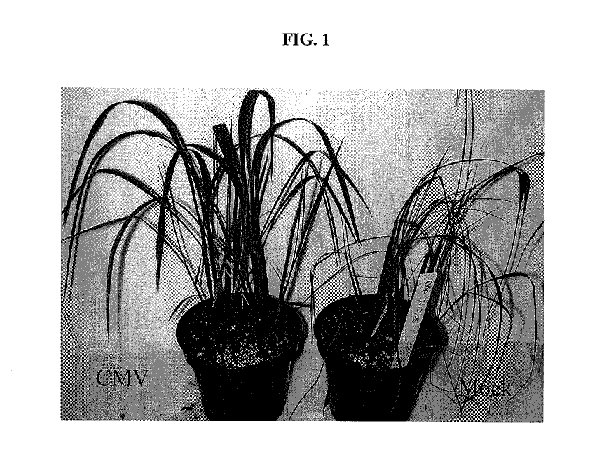

FIG. 2: Comparison of drought symptoms in mock- and BMV-inoculated rice plants at 6 days after watering.

FIG. 3: Comparison of drought symptoms in mock- and TMV-inoculated N. benthamiana plants at 8 days after watering.

### DETAILED DESCRIPTION OF THE INVENTION

Described herein are methods and compositions for improving stress tolerance in plants. The methods enhance the ability of plants to withstand stress such as drought. Surprisingly, it was found that systemic infection of a host plant by a symptomatic or asymptomatic plant virus improves the growth and/or survival of the host under drought conditions. As used herein, “drought” refers to a condition in which a plant is subjected to osmotic stress or reduced water potential.

Although plant viruses were initially identified and studied based on their ability to cause disease, as disclosed herein this previous paradigm represents an incomplete picture of the virus-host relationship. Rather, it is demonstrated that mutualistic relationships may be established between a plant pathogenic virus and its symptomatic or asymptomatic host plant under drought stress. In this instance, the appearance of drought symptoms may be delayed and/or their severity may be reduced in a virus-infected plant as compared to uninfected plants of the same species, line, or cultivar, under the same environmental conditions.

The present invention provides methods for improving drought (dehydration) tolerance in plants subject to water limiting conditions comprising introducing a plant virus into a plant. The plant virus may induce a viral disease symptom upon its introduction, yet infection results in enhanced drought tolerance. Alternatively, no symptoms of viral disease may be induced, however drought tolerance is nevertheless enhanced. Drought tolerance may be assessed by comparing a virus infected plant with a non-infected plant of the same genotype, variety, or cultivar grown under the same conditions of drought or limited water availability. One or more symptoms may be compared between infected and uninfected plants, including, for instance, wilting, growth arrest, leaf rolling, leaf distortion, leaf drop, leaf scorch, stem or twig dieback, photosynthetic efficiency, flowering, and yield level.

Drought tolerant plants demonstrate fewer or less severe symptoms of stress caused by lack of available water. Drought conditions, or lack of available water, may be assessed by comparing the amount of water required for plant growth or maturation with the amount of water available to a plant. Drought conditions may be caused by lack of rainfall or irrigation, relative to the amount of water used internally or transpired by a plant. Physiological parameters such as water potential may be measured to quantify a plant's level of water stress. Phenotypic assessment of symptoms described above may be utilized to determine whether, and to what extent, a plant is suffering from drought. Alternatively, biochemical or nucleic acid based assays may be performed to assess a plant's response to water limitation.

A plant or plant tissue may be inoculated or infected with a virus or infectious viral material by any suitable method for delivering the material to the plant, such as by mechanical inoculation or by spraying, by injection or infiltration, by grafting, by seed or pollen transmission, or by vector transmission (e.g. by an insect or nematode vector), among others. Inoculation may be performed, for example, into a wound or in the presence of an abrading agent. Spray-inoculation may also be employed, optionally in conjunction with abrasion of plant tissues.

The infectious material may comprise purified, partially purified, or unpurified nucleic acids or viral particles, including an infectious viral DNA or cDNA clone, infectious RNA transcript(s), virions, sap from infected plants, ground leaves, or other tissue or tissue extract from an infected plant. By “infectious”, it is meant that the material allows for viral replication within a plant cell.

The infectious material may be suspended or diluted in a physiologically acceptable carrier, solvent, or diluent, such as a phosphate buffer solution (e.g. at a pH of 7.0-7.4), or other dispersion media, and the like. An inoculum-containing solution may also be prepared in water suitably mixed with a surfactant, such as hydroxypropylcellulose, and/or an abradant such as Carborundum™ or silicon carbide. Dispersions also can be prepared in a solution comprising glycerol, or a polyol and mixtures thereof. The infectious material may be in the form of a solution, an aerosol, a dispersion, a powder, or the like. A virus strain selected for inoculation may be genetically engineered with a coding or non-coding sequence, for instance to allow for specific detection of the inoculated strain, for instance by nucleic acid amplification or hybridization, by antigen detection, or by activity of a marker protein such as GFP (Green Fluorescent Protein).

The tested plant virus strains resulted in improved drought tolerance under the conditions used. Thus, the invention is not limited to any specific plant virus strain. In certain embodiments, the virus utilized for plant infection may be an RNA virus such as a Cucumovirus, a Tobamovirus, a Tobravirus, or a Bromovirus. In particular embodiments, the virus may be a strain of: Cucumber Mosaic Virus (CMV), Tobacco Mosaic Virus (TMV), Tobacco Rattle Virus (TRV), or Brome Mosaic Virus (BMV), among others. In other embodiments, other RNA viruses may be used and in a further embodiment a DNA virus is used according to the invention.

In one embodiment of the invention, the plant may be a monocot crop plant, such as rice. In another non-limiting embodiment of the invention, the plant may be, for example, a dicot crop plant such as tobacco, cucumber, tomato, pepper, beet, watermelon, zucchini. The plant may also be an ornamental plant, such as poinsettia.

The invention also provides a method for altering water consumption by a plant, wherein a virus infected plant requires or consumes less water than an uninfected plant of the same genotype, variety, or cultivar in order to achieve comparable survival, growth, flowering, and/or production of harvestable material. The invention further provides a method to reduce osmotic stress damage in a plant, by selecting a plant under osmotic stress or at risk for osmotic stress, and infecting the plant with a plant virus, such that the plant displays fewer, less severe, and/or delayed symptoms of water deficiency relative to an uninfected plant.

Techniques for nucleic acid and protein detection may find use in certain embodiments of the invention. For example, such techniques may find use in testing for the presence of a virus in a plant that may be infected with a virus.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Viruses and Plant Materials

The Russian strain of Brome mosaic virus (BMV) and the U1 and mutant MIC-1,3-strains of Tobacco mosaic virus (TMV) were used (Shintaku et al., 1996) for BMV and TMV inoculations, respectively. For Tobacco rattle virus (TRV) infection, a cDNA clone derived from, and containing a portion of the GFP gene, was used (Ryu et al., 2004). The Fny strain of Cucumber mosaic virus (CMV), previously described (Xu et al., 2004), was used for CMV inoculation. Inoculum consisted of purified virus (CMV) or sap from infected plants (BMV, TMV and TRV). For CMV or TMV inoculum was prepared with purified TMV particles in pH 7.2 PBS (0.05 mM) at a concentration of 200 ng/μl and 10 μl of inoculum was placed on the leafs of tested plants. For sap inoculation infected N. benthamania plants were ground in PBS buffer (pH 7.2, 50 mM) and 40 μl sap was inoculated onto leafs of tested plants by mechanical grinding.

Seedlings at the 2-3 leaf stage were used for inoculation of beet (Beta vulgaris cv. Detroit Dark Red), pepper (Capsicum annuum cv. Marango), watermelon (Cucumis lanatus cv. Crimson Sweet), cucumber (Cucumis sativus cv. National Pickling), tomato (Lycopersicon esculentum cv. Rutgers), Lycopersicon hirsutum, rice (Oryza sativa cv. IR-8) and zucchini (Cucurbita pepo, cv. Elite). One month old seedlings were used for inoculation of Nicotiana benthamiana and of tobacco (N. tabacum cv. Xanthi nc for CMV inoculation, and cv. Xanthi nn for TMV inoculation). For TMV infections, N. benthamiana was inoculated with the MIC-1-3-mutant of U1-TMV because U1-TMV is lethal, and tobacco was inoculated with the U1-strain of TMV.

### Example 2

### Drought Treatment

For each plant species, eight to fifteen individual plants were inoculated with buffer or viral inoculum. After inoculation, the plants were grown in four separate growth rooms. The temperature ranges of these growth rooms were 20° C. (night) 26° C. (day) for TMV and CMV rooms, 19° C. (night) 24° C. (day) for the BMV room, and 18° C. (night) 23° C. (day) for the TRV room, with 16 hour days. Eight days (watermelon, cucumber, tomato, pepper, C. amaranticolor and L. hirsutum plants) or two weeks (N. benthamiana and tobacco) post-inoculation plants were bottom watered for two days to saturate the soil, and then moved to dry flats where water was withheld. The drought-treated plants were photographed with a COOLPIX990 digital camera (Nikon, Melville, N.Y., USA) once every day (days after withholding water; daw) from the onset of drought symptoms until the death of the mock-inoculated plants.

### Example 3

### Infection of Plants with Cucumber Mosaic Virus (CMV)

Eleven different plant species were infected with the broad host range virus Cucumber mosaic virus (CMV), strain Fny (Roossinck, 1998) (Table 1). These plant species included rice, Oryza sativa, which has not been reported previously as a host of CMV. Inoculum consisted of purified CMV. Table 1 lists the plant species inoculated with CMV and their response to drought conditions. The appearance of drought symptoms in virus-infected plants was delayed by 2-5 days compared with mock-inoculated plants.

TABLE 1
Time, in days, of the onset of drought symptoms
and plant death after withholding water for
mock-inoculated and CMV-infected plants.

| | | Appearance of | | Occurrence of | |
|---|---|---|---|---|---|
| | | symptoms | | plant death | |
| | | Mock | CMV | Mock | CMV |
| Plant species | Common name | (daw) | (daw) | (daw) | (daw) |
| Beta vulgaris | Beet | 5 | 8 | 8-9 | 11-12 |
| Capsicum annum | Pepper | 2 | 6-8 | 5-6 | 11-13 |
| Chenopodium | Lambsquarters | 3 | 6 | 6 | 8 |
| amaranticolor | | | | | |
| Cucumis lanatus | Watermelon | 5 | 9-10 | 9 | 11-12 |
| Cucumis sativus | Cucumber | 3 | 6 | 5 | 9-10 |
| Cucurbita pepo | Zucchini | 3 | 6 | 9-10 | 12 |
| Lycopersicon | Tomato | 1 | 3 | 2-3 | 5-7 |
| esculentum | | | | | |
| Lycopersicon | — | 7 | 10-11 | 9-10 | 12-13 |
| hirsutum | | | | | |
| Nicotiana | — | 2 | 6 | 15-16 | 18-19 |
| benthamiana | | | | | |
| Nicotiana tabacum | Tobacco | 2 | 4-5 | 7-10 | 8-11 |
| Oryza sativa | Rice | 6 | 8-9 | 9 | 11-14 |

### Example 4

### Identification of Systemic Infection of CMV

To determine if rice supports CMV infection, the systemic (i.e. non-inoculated leaves) leaf tissues of buffer- or CMV-inoculated rice and tomato plants (positive control) were harvested, and total RNA was extracted as previously described (Xu et al., 2004). Five μg of total RNA was used for RT-PCR amplification of CMV RNA3 using forward primer 5′ GGATGCGCGCTGATAATGCT 3′ (SEQ ID NO:1) and reverse primer 5′ CCGAAGGAATTCCGAAGAAACCTAGG 3′ (SEQ ID NO:2) primers. The amplified fragment was gel extracted and analyzed by sequence analysis. Following inoculation, asymptomatic rice plants were found to support CMV replication.

To determine if CMV moves out of the inoculated leaves of C. amaranticolor, the inoculated leaves (positive control), the stem above the CMV-inoculated leaves and the non-inoculated upper leaves of buffer- or CMV-inoculated C. amaranticolor were harvested and total RNA was extracted and analyzed as described above.

### Example 5

### N. benthamiana Inoculations

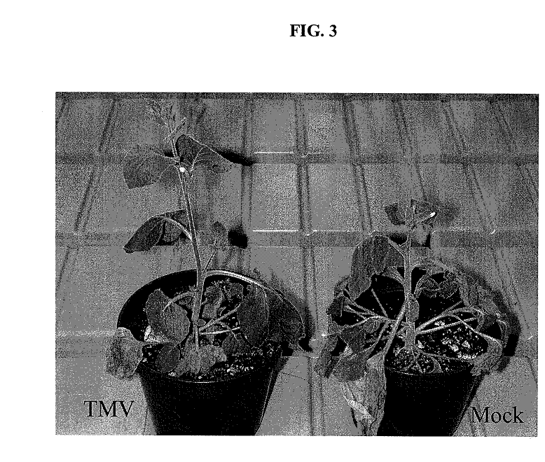

Nicotiana benthamiana is a common host for the four RNA viruses used in this study. Thus, N. benthamiana plants were inoculated with these viruses to compare their effects on drought tolerance. The appearance of drought symptoms in virus-infected plants was delayed by 2-5 days compared with mock-inoculated plants (Table 2). The TMV-infected plants exhibited slow but perceptible growth while the mock-inoculated plants suffered growth arrest soon after the withholding of water began (FIG. 3).

TABLE 2
Time, in days, of the onset of drought symptoms
and plant death after withholding water between
mock- and virus-infected N. benthamiana plants.

| | | | | | MIC-1,3- | | | |
|---|---|---|---|---|---|---|---|---|
| | BMV (daw) | | CMV (daw) | | TMV (daw) | | TRV (daw) | |
| | Mock | BMV | Mock | CMV | Mock | TMV | Mock | TRV |
| Appearance | 4 | 6 | 2 | 6 | 4 | 7 | 3 | 7 |
| of drought | | | | | | | | |
| symptoms | | | | | | | | |
| Occurrence | 21-22 | 22-24 | 15-16 | 18-20 | 18-19 | 21-22 | 16-18 | 19-22 |
| of plant | | | | | | | | |
| death | | | | | | | | |

### Example 6

### Inoculation of Rice and Tobacco

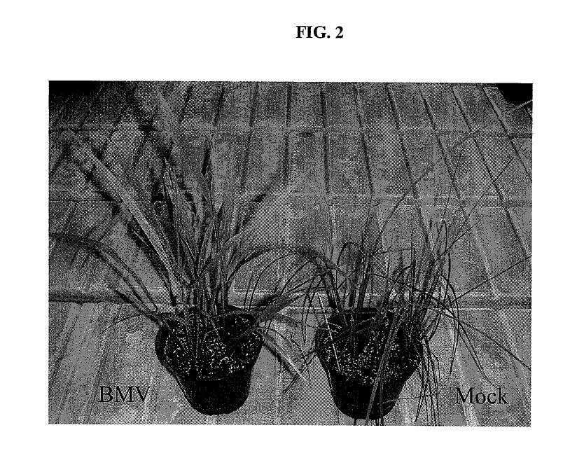

Rice seedlings were inoculated with BMV as described above. BMV-infected rice showed the first sign of drought stress (rolled leaves) 9-10 days after withholding water (daw), by which time mock-inoculated rice plants were completely wilted (FIG. 2). Tobacco seedlings (N. tabacum genotype nn) were inoculated with TMV as described above. TMV-infected tobacco plants survived with turgid stems and green tips for 50-55 daw, while mock-inoculated plants died more than twenty days earlier.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bohnert et al., 1995. Plant Cell 7:1099-1111.
Bray, 1997. Trends Plant Sci. 2:48-54.
Gage et al., 2001. Mol. Microbiol. 41:775-785.
Roossinck, M. J. and P. S. White. 1998. in Plant Virology Protocols G. D. Foster, S. C. Taylor, Eds. (Humana Press, Totowa, N.J.), vol. 81, pp. 189-196.
Ryan, F. 2004. J R Soc. Med. 97:560
Ryu, et al., 2004. Plant J 40:322-331.
Schardl, et al. 2004. Ann. Rev. Plant Biol. 55:315-340.
Shintaku et al., 1996. Virology 221:218-225.
Stasiak, et al., 2005. J Insect Physiol. 51:103-115.
Webb, B A, in The Insect Viruses L. K. Miller, L. A. Ball, Eds. (Plenum Publishing Corporation, New York, 1998) pp. 105-139.
Whenham et al., 1986. Planta 168:592-598.
Wollenweber, et al., 2005. Cur. Opinion. in Plant Biol. 8:337-341.
Xu et al, 2004. Proc. Natl. Acad. Sci. USA 101:15805-15810.

What is claimed is:

1. A method for enhancing drought tolerance in a plant comprising:
(a) identifying a plant in need of increased drought tolerance; and
(b) infecting the plant with a plant virus or infectious plant viral material that increases the drought tolerance in the plant as compared to an uninfected plant of the same genotype when grown under the same conditions.

2. The method of claim 1, wherein the virus produces a disease symptom in the plant.

3. The method of claim 1, wherein the virus does not produce a disease symptom in the plant.

4. The method of claim 1, wherein step (a) comprises identifying a plant exhibiting drought stress.

5. The method of claim 1, wherein step (a) comprises identifying a plant in a growing environment under drought conditions.

6. The method of claim 1, wherein the plant is selected from the group consisting of rice, beet, cucumber, zucchini, watermelon, tomato, tobacco, and Nicotiana sp.

7. The method of claim 6, wherein the plant is rice (Oryza sativa).

8. The method of claim 7, wherein the virus is Cucumber Mosaic Virus (CMV).

9. The method of claim 1, wherein the virus is selected from the group consisting of: Cucumoviruses, Tobamoviruses, Tobraviruses, and Bromoviruses.

10. The method of claim 9, wherein the virus is selected from the group consisting of Cucumber Mosaic Virus, Tobacco Mosaic Virus, Tobacco Rattle Virus, and Brome Mosaic Virus.

11. The method of claim 1, wherein the infectious material comprises a plant virus virion, an RNA transcript, or a plant virus cDNA clone.

12. The method of claim 1, wherein the plant is infected with a virus by a method selected from the group consisting of: mechanical inoculation, spraying, injection, infiltration, grafting, seed or pollen transmission, and vector transmission.

13. The method of claim 12, wherein the method for virus infection is mechanical inoculation.

14. The method of claim 12, wherein the virus is selected from the group consisting of Cucumber Mosaic Virus, Tobacco Mosaic Virus, Tobacco Rattle Virus, and Brome Mosaic Virus.

15. A method of reducing osmotic stress damage in a plant, comprising the steps of:
(a) identifying a plant under osmotic stress; and
(b) infecting the plant with a plant virus.


SEQUENCE LISTING (2 sequences)

SEQ ID NO: 1 - DNA, 20 bp, Artificial
  DNA primer
ggatgcgcgctgataatgct

SEQ ID NO: 2 - DNA, 26 bp, Artificial
  DNA primer
ccgaaggaattccgaagaaacctagg